(19) United States
(12) Patent Application Publication
(10) Pub. No.: US 2025/0205276 A1
(43) Pub. Date: Jun. 26, 2025

(54) COMPOSITIONS AND METHODS OF TREATMENT OF DISEASE USING COMBINATION OF A NITRODILATOR AND A NITROGEN OXIDE COMPOUND

(71) Applicant: LOMA LINDA UNIVERSITY, Loma Linda, CA (US)

(72) Inventors: Arlin B. Blood, Loma Linda, CA (US); Taiming Liu, Loma Linda, CA (US)

(73) Assignee: LOMA LINDA UNIVERSITY, Loma Linda, CA (US)

(21) Appl. No.: 18/845,488

(22) PCT Filed: Mar. 13, 2023
(86) PCT No.: PCT/US2023/064264

§ 371 Date: Sep. 10, 2024

Related U.S. Application Data
(60) Provisional application No. 63/269,223, filed on Mar. 11, 2022.

(51) Int. Cl. A61K 33/00; A61K 31/17; A61K 31/198; A61K 31/21; A61K 31/34; A61K 38/51

(52) U.S. Cl. CPC A61K 33/00; A61K 31/17; A61K 31/198; A61K 31/21; A61K 31/34; A61K 38/51

(57) ABSTRACT

Provided here are compositions and methods for treatment of vascular conditions or respiratory conditions by administering a therapeutically effective amount of a nitrogen oxide compound and a nitrodilator. The nitrogen oxide compound can be nitrite, one or more of a number of nitro-group containing compounds, a nitrate-group containing compound, an NO donor, or an endogenous NO producing amino acid. The nitrodilator can be one or more of nitroglycerin, isosorbide dinitrate, isosorbide mononitrate, nitroprusside, amyl nitrite, nitrosothiols, or dinitrosyl iron complexes.

### GOVERNMENT SUPPORT

This invention was made with government support under grant numbers: HL095973 and HD083132 awarded by National Institutes of Health. The government has certain rights in the invention.

### TECHNICAL FIELD

The disclosure relates to compositions and methods of treatment of disease using a combination of a nitrodilator and a nitrogen oxide compound.

### BACKGROUND

Nitric oxide (NO) is a ubiquitous signaling messenger in biological systems. As a chemically reactive free radical, NO has a relatively short half-life. For many years, the effects of endogenous NO were believed to be only paracrine, as it is inactivated in blood within milliseconds, which would preclude any endocrine, that is far reaching and long lasting, signaling activity. However, early studies of the use of inhaled NO gas to treat pulmonary hypertension surprisingly revealed vasodilation of downstream systemic vascular beds despite the fact that it takes blood more than ten seconds to travel from pulmonary capillaries to the peripheral resistance vessels. NO metabolism in biological matrices is a complex web of reactions leading to a wide array of products that include nitrite, nitrate, nitrotyrosines, and various molecules with the NO bound to thiols or iron. A subset of these NO-containing compounds, which include S-nitrosothiols (SNO), dinitrosyl iron complexes (DNIC) and nitroglycerin (NTG), dilate vessels via activation of soluble guanylyl cyclase (sGC) in vascular smooth muscle cells. A class of these compounds, defined here as ‘nitrodilators’, have vasodilatory potency comparable to or even greater than that of NO itself, despite low membrane permeability. Although these compounds are often considered to achieve vasodilation by releasing their NO moiety to enter the vascular smooth muscle cell and activate sGC, overwhelming experimental evidence contradicts this understanding.

### SUMMARY

Provided here are systems and methods to address these shortcomings of the art and provide other additional or alternative advantages. The disclosure herein provides one or more embodiments of systems and methods for treatment of vascular conditions or local tissue NO deficiencies by administering a therapeutically effective amount of a nitrogen oxide compound and a nitrodilator. The combination of nitrodilator and the nitrogen oxide compound can be administered as either a fixed-dose combination or as separate formulations. The nitrodilator can be one or more of nitroglycerin, isosorbide dinitrate, isosorbide mononitrate, nitroprusside, amyl nitrite, nitrosothiol, or dinitrosyl iron complexes. The nitrogen oxide compound can be one or more of a nitro-group containing compound, a nitrate-group containing compound, an NO donor, and an endogenous NO producing amino acid. The nitro-group containing compound can include a nitrite salt, a D-NG-Nitro arginine methyl ester, or any other nitro-group containing low molecular weight molecules, or proteins, or lipids. The nitrate-group containing compound can include a nitrate salt or any nitrate-group containing low molecular weight molecules, or proteins, or lipids. The NO donor can be a NONOate or an hydroxamic acid such as deferoxamine, acetohydroxamic acid, hydroxyurea. A NONOate is a compound with an amine functional group, a bridging NO″ group, and a terminal nitrosyl group, which releases NO under certain biological conditions. The endogenous NO producing amino acid can be arginine or citrulline or combinations thereof.

Embodiments include methods of treating a vascular condition or a respiratory condition in a human subject by administering to the subject a therapeutically effective amount of a stimulator of soluble guanylyl cyclase (sGC stimulator) in addition to a therapeutically effective amount of a nitrogen oxide compound and a nitrodilator. In certain embodiments, the sGC stimulator is riociguat or vericiguat. Embodiments include methods of treating a vascular condition or a respiratory condition in a human subject by administering a therapeutically effective amount of an activator of soluble guanylyl cyclase (sGC activator) in addition to a therapeutically effective amount of a nitrogen oxide compound and a nitrodilator. In certain embodiments, the sGC activator is runcaciguat, cinaciguat, ataciguat, or kynurenine. Embodiments include methods of treating a vascular condition or a respiratory condition in a human subject by administering to the subject a cGMP (cyclic guanosine monophosphate)-phosphodiesterase inhibitor along with a therapeutically effective amount of a nitrogen oxide compound and a nitrodilator. In certain embodiments, the cGMP-phosphodiesterase inhibitor is sildenafil, vardenafil, or tadalafil.

Embodiments include methods of treating a vascular condition or a respiratory condition in a human subject by administering to the subject a therapeutically effective amount of a nitrogen oxide compound and nitric oxide administered via inhalation. In certain embodiments, the nitric oxide is administered via inhalation together with a nebulized thiol. The nebulized thiol can be L-cysteine or glutathione.

Embodiments include methods of treating a vascular condition or a respiratory condition in a subject by administering to the subject a therapeutically effective amount of a first nitrodilator in a first formulation to activate a nitrodilator-activatable intracellular NO store (NANOS) and a second nitrodilator in a second formulation to contribute to the NANOS. In certain embodiments, the first nitrodilator is nitroglycerin and the first formulation is a sublingual tablet or a trans-lingual spray. In certain embodiments, the second nitrodilator is nitroglycerin and the second formulation is a nanoliposome. In certain embodiments, the second nitrodilator is a dinitrosyl iron complex (DNIC), S-nitroso-glutathione (GSNO), or heme-NO and the second formulation is a nanoliposome.

Methods of treating a vascular condition or a respiratory condition in a subject include administering to the subject a therapeutically effective amount of a nitrogen oxide compound and a cGMP-phosphodiesterase inhibitor. The nitrogen oxide compound can be a nitrate-group containing compound or a nitrite compound. The cGMP-phosphodiesterase inhibitor can be sildenafil, vardenafil, or tadalafil. The vascular condition can be erectile dysfunction.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. Embodiments are illustrated by way of example and not by way of limitation in the accompanying drawings.

FIG. 1A (Prior Art) is an illustration of production pathways and previously proposed vasodilatory mechanisms of three prominent NO storage forms: nitrite, DNICs, and SNOs. All three have been proposed to mediate vasodilation via various routes of conversion to free NO. DNICs and SNOs, most of which are membrane impermeable, have been proposed to release free NO outside the vascular smooth muscle cell, with the exception of possible selective transport of SNOs such as S-nitrosocysteine (L-cysNO) or L-cysNO-glycine (L-cysNO-gly) across the plasma membrane via the L-type amino acid transporter (LAT) or dipeptide transporters (PEPT2), respectively. Handoff of NO from extracellular SNOs to membrane thiols via transnitrosylation with subsequent transport of the NO to the cytoplasm has also been proposed. FIG. 1B is a representation of the structure of a mononuclear dinitrosyl iron complex.

FIG. 2 is an illustration of mechanism of the nitrodilator-activatable intracellular NO store (NANOS). Nitrite (NO₂⁻) and nitrodilators (NTG, SNOs, and DNICs; latter two can be endogenous NO metabolites) circulate systemically. NO₂⁻ contributes to a store of NO bioactivity within the vascular smooth muscle cell. Extracellular nitrodilators cause sGC-dependent vasodilation by stimulating release of an NO equivalent (NOe) from the NANOS.

FIGS. 3A-3F demonstrate MDNICs as a candidate of the NANOS (n=5). FIG. 3A is an illustration of the contribution of the NANOS by nanoliposomes containing MDNIC. FIG. 3B is a graphical representation of the diameter of nanoliposomes that enclose glut-MDNIC, with a mean concentration of 0.68 μM. FIG. 3C is a graphical representation of the electron paramagnetic resonance (EPR) intensity of the control and MDNIC-containing nanoliposomes. Incubation with MDNIC-containing nanoliposomes introduces MDNIC into the arteries as measured by electron paramagnetic resonance spectroscopy. FIG. 3D is a photographical representation of the uptake of nanoliposomes tagged with Texas Red into the arterial wall as evidenced by fluorescence image.

FIG. 3E is a graphical representation of the response of arteries following incubation with nanoliposomes containing MDNIC, demonstrating a reversal of loss of vasodilatory response that occurs following repeated exposure to S-nitrosoglutathione (GSNO), while nanoliposomes encapsulated with GSNO or buffer (empty) do not. FIG. 3F is a graphical representation of the response of arteries following incubation with nanoliposomes containing MDNIC, demonstrating that the incubation does not alter NO-mediated vasodilation for all three samples.

FIGS. 4A-4D demonstrate the mobilization of the NANOS. FIG. 4A is an illustration of the protocol. A sheep carotid artery (length 8 cm) was incubated with 10 μM N¹⁵-nitrite in DMEM under 10.5% O₂ for 6 h to load the vessel with N¹⁵-NOx (NO metabolites). FIG. 4B is an illustration of the prepared vessels. After extensive washes, the arteries were well sealed and incubated for 2 h with 3 ml 100 μM oxyhemoglobin in HEPES buffer on the extraluminal side and 5 μM GSNO or control (0.7 ml HEPES buffer containing 0.1 mM DTPA) on the luminal side. FIG. 4C is a graphical representation of the response of the arteries exposed to GSNO. The N¹⁵-NOx levels were lower (p=0.0206) in the homogenate of arteries exposed to GSNO. FIG. 4D is a graphical representation of the response of the arteries exposed to intraluminal GSNO. The N¹⁵-NOx in extraluminal buffer was higher (p=0.0400) in the arteries exposed to intraluminal GSNO. N¹⁵-NOx was measured by GC-MS. The results are consistent with intraluminal GSNO stimulating an efflux of NOx from the vascular smooth muscle as manifested by a decrease in intracellular NOx and an increase in extracellular NOx.

FIGS. 5A-5D demonstrate the HNO-like properties of the NO equivalent released from the NANOS. Isolated sheep mesenteric arteries were denuded of endothelium and contracted with 10 μM 5-HT (n=5). FIG. 5A presents examples of relaxation traces (one animal, six arterial strips) of NTG in the absence and presence of an sGC inhibitor (ODQ) (10 μM) or superoxide dismutase (SOD1) (1000 U/ml, converts HNO into NO), or the extracellular NO scavenger CPTIO (200 μM), or SOD1 and CPTIO together. All traces were normalized for comparison of extent of relaxation.

FIG. 5B is a graphical representation of the amplification of the area in the red circle in ‘A’. FIG. 5C is a graphical representation of the effects of SOD1, CPTIO, SOD1+CPTIO, and ODQ on relaxation in response to Nitroglycerin. FIG. 5D is a graphical representation of the effects of SOD1, CPTIO, and SOD1+CPTIO on relaxation in response to nitrosoglutathione (GSNO). SOD1, via putative conversion of HNO to NO, enables the inhibitory effects of CPTIO on relaxation caused by NTG and GSNO.

### DETAILED DESCRIPTION

Nitroglycerin is a nitrodilator that is commonly prescribed for the treatment of angina. It works by vasodilating the coronary arteries, thus increasing blood flow to the myocardium, and by vasodilating the systemic venous vessels, thereby decreasing venous return and ventricular preload resulting in decreased cardiac work. A significant hindrance to the clinical use of nitroglycerin is that a tolerance to its vasodilatory effects develops after repeated use. The mechanism for this tolerance has not yet been characterized despite much study. The tolerance may be due to depletion of the vascular intracellular NO stores. Currently, nitrodilators such as nitroglycerin are prescribed alone for treatment of angina. Provided here are methods of co-administration of nitro-group containing compounds such as nitrite, which would replenish the intracellular store and thus attenuate the tolerance that develops to repeated treatment with nitrodilators, including NTG.

Disclosed here are methods of co-administration of one or more nitro-group-containing chemicals together with nitrodilators to mitigate the tolerance and loss of therapeutic effects that develop in subjects that require frequent dosing. Nitro-group-containing chemicals are generally considered as stable and thus in-active and safe compounds. This class of compounds gradually release nitric oxide (NO) via Fenton chemistry-related mechanisms or others, and augment vasodilation of blood vessels, contributing to decreased blood pressure. Nitrite, the endogenous anion form of nitro-group, also releases NO via Fenton chemistry-related mechanisms. In some embodiments, nitro-group containing compounds such as nitrite are provided as a useful supplement for beneficial cardiovascular effects via NO-mediated effects on the vasculature. Vascular smooth muscle contains intracellular stores of NO bioactivity that contribute to vasodilation. The intracellular store can be activated by the presence of extracellular compounds, classified herein as nitrodilators, such as nitrosothiols and nitroglycerin. Repeated activation results in depletion of the intracellular store and impaired vasodilatory function. In addition, in experiments with isolated arteries and living animals, the intracellular store can be replenished by the administration of nitro-containing chemicals that act as NO donors. Such treatment restores the vasodilatory effects of nitrodilators such as NTG and nitrosothiols. Disclosed herein are embodiments of methods for treatment of vascular conditions by administering a therapeutically effective amount of a nitrogen oxide compound and a nitrodilator, such as NTG.

Methods of treating vascular or respiratory conditions in a human subject include administering a therapeutically effective amount of a nitrogen oxide compound and a source of exogenous NO or an agent that leads to production of endogenous NO. For example, exogenous NO can be inhaled NO. In certain embodiments, the NO can be inhaled along with a nebulized thiol, such as L-cysteine or glutathione.

Methods for treatment of vascular conditions or respiratory conditions include administering a therapeutically effective amount of two different formulations of a nitrodilator. Certain methods of treating a vascular condition or a respiratory condition in a human subject include administering to the subject a therapeutically effective amount of a nitrodilator to activate the NANOS in a first formulation and a nitrodilator to contribute to the NANOS in a second formulation. For example, in an embodiment, NTG is administered as a sublingual tablet or a translingual spray along with another administration of NTG as a nanoparticle. In an embodiment, the nanoparticle can be liposome-encapsulated NTG. In certain embodiments, the first formulation can be a sublingual tablet or a trans-lingual spray and the second formulation is liposome-encapsulated nitroglycerin, DNIC, GSNO, or heme-NO. The therapeutically effective amount of a nitrogen oxide compound or the nitrodilator can be trapped, encapsulated, and/or absorbed within liposomes, lipidic or polymeric nanoparticles, vesicles, or microemulsions to be used as delivery vehicles. Biopolymers, such as polylactide, polyglycolide, poly(lactide-co-glycolide), polyhydroxybutyrate, polycaprolactone, and polydioxanone can be used to make subunit nanoparticles and/or composite nanoparticles. Natural hydrophilic polysaccharides, such as hyaluronic acid or inulin, are useful for producing subunit nanoparticles and/or composite nanoparticles. In some embodiments, a nitrogen oxide compound or the nitrodilator a compound described herein can be coupled to a polymer used in the nanoparticle, for example a polystyrene particle, PLGA particle, PLA particle, or other nanoparticle.

Particular vascular conditions that are treatable and/or preventable by the compositions disclosed herein include, but are not limited to, coronary and peripheral arterial diseases, asthma, chronic rejection, vasculopathy associated with diabetes, circulatory congestive states, peripheral edema, scleroderma, glaucoma, systemic hypertension, pulmonary hypertension, wound healing, anal fissures, vulvodynia, erectile dysfunction, post-hemorrhagic vasospasm, preeclampsia, Raynaud's phenomenon, and heparin overdose. Particular respiratory conditions that are treatable and/or preventable by the compositions disclosed herein include, but are not limited to, apnea, chronic obstructive pulmonary disease, and pulmonary hypertension.

The combination of a nitrodilator and the nitrogen oxide compound can be administered as either a fixed-dose combination or as separate formulations. The nitrogen oxide compound can be one or more of a nitro-group containing compound, a nitrate-group containing compound, an NO donor, and an endogenous NO producing amino acid. The nitro-group containing compound can include a nitrite salt, a N5-[imino (nitroamino)methyl]-D-ornithine (D-NG-Nitro arginine methyl ester), or any other nitro-group containing low molecular weight molecules, or proteins, or lipids. The nitrate-group containing compound can include a nitrate salt or any nitrate-group containing low molecular weight molecules, or proteins, or lipids. The NO donor can be a NONOate or an hydroxamic acid. A NONOate is a compound with an amine functional group, a bridging NO″ group, and a terminal nitrosyl group, which releases NO under certain biological conditions. An hydroxamic acid includes, but is not limited to, deferoxamine, acetohydroxamic acid, and hydroxyurea. The endogenous NO producing amino acid can be arginine or citrulline or combinations thereof.

The present disclosure relates to compositions for the treatment of vascular conditions or respiratory conditions by increasing the effective amount of NO available to the affected cells and/or tissue. The term “therapeutically effective amount” refers to an amount of a compound that, when administered to a subject suffering from a condition, will have the intended therapeutic effect, e.g., alleviation, amelioration, palliation or elimination of one or more manifestations of the condition in the subject. The therapeutically effective amount will vary depending upon the subject and the condition being treated, the weight and age of the subject, the severity of the condition, the salt, solvate, or derivative of the active drug portion chosen, the particular composition or excipient chosen, the dosing regimen to be followed, timing of administration, the manner of administration and the like. The full therapeutic effect does not necessarily occur by administration of one dose, and may occur only after administration of a series of doses. Thus, a therapeutically effective amount may be administered in one or more administrations.

As used herein, “treatment” or “treating” is an approach for obtaining a beneficial or desired result, including clinical results. For purposes herein, beneficial or desired results include but are not limited to inhibiting and/or suppressing the onset and/or development of a disease or condition that is responsive to the compositions described herein or reducing the severity of such disease or condition, such as reducing the number and/or severity of symptoms associated with the disease or condition, increasing the quality of life of those suffering from the disease or condition, decreasing the dose of other medications required to treat the disease or condition, enhancing the effect of another medication an individual is taking for the disease or condition and prolonging survival of individuals having the disease or condition. As used herein, “preventing” refers to reducing the probability of developing a disorder or condition in an individual who does not have, but is at risk of developing a disorder or condition.

In some embodiments, the therapeutic dose of a nitrodilator, or pharmaceutically acceptable salt or solvate thereof, administered to the subject is sufficient to provide an average serum concentration of about 100 μg/mL to about 1 μg/mL, or any subrange or subvalue there between. In an embodiment, a nitrodilator like NTG is administered at a dose ranging from 0.1 mg to about 0.6 mg each time. In an embodiment, a nitrodilator like isosorbide mononitrate is administered at a dose ranging from 10 mg to about 300 mg, and can include about 120 mg per tablet. In an embodiment, the dose of nitrodilator, or pharmaceutically acceptable salt or solvate thereof, administered to the subject is about 0.1 mg to 0.6 mg. In some embodiments, the therapeutic dose of the nitrogen oxide compound ranges from about 1 mg to about 10 g. In some embodiments, the nitrodilator and the nitrogen oxide compound are administered to the subject at a ratio ranging from 1:1 to 1:33, or from 1:1 to 1:333, or from 1:1 to 1:3333, or from 1:1 to 1:33333. In some embodiments, the nitrodilator and a nitrate compound are administered to the subject at a ratio ranging from 1:1 to 1:34, or from 1:1 to 1:333, or from 1:1 to 1:3333, or from 1:1 to 1:33333. The nitrate compound can be a pharmaceutically acceptable salt, such as a sodium salt, or solvate thereof. In some embodiments, the nitrodilator and a nitrite compound are administered to the subject at a ratio ranging from 1:1 to 1:33, or from 1:1 to 1:333, or from 1:1 to 1:3333. The nitrite compound can be a pharmaceutically acceptable salt, such as a sodium salt, or solvate thereof. In some embodiments, a therapeutically effective amount of a nitrogen oxide compound and nitroglycerin is administered as a fixed-dose combination. In some embodiments, a therapeutically effective amount of a nitrogen oxide compound and nitroglycerin is administered as a free-dose combination.

The term “about” when used before a numerical designation, e.g., temperature, time, amount, concentration, and such other, including a range, indicates approximations which may vary by (+) or (−) 10%, 5% or 1%, or any subrange or subvalue there between.

Any route of administration, such as oral, topical, subcutaneous, peritoneal, intra-arterial, inhalation, vaginal, rectal, nasal, introduction into the cerebrospinal fluid, or instillation into body compartments can be used. The compositions herein can be administered by direct blood stream delivery, e.g. sublingual, intranasal, or intrapulmonary administration. Administration can be via transdermal patch, gum, lozenge, sublingual tablet, intranasal, intrapulmonary, oral administration, or other administration. “Pharmaceutically acceptable salt” refers to pharmaceutically acceptable salts, including pharmaceutically acceptable partial salts, of a compound, which salts are derived from a variety of organic and inorganic counter ions well known in the art and include, by way of example only, hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, oxalic acid and the like, and when the molecule contains an acidic functionality, include, by way of example only, sodium, potassium, calcium, magnesium, ammonium, tetraalkylammonium, and the like.

As used herein the term “solvate” is taken to mean that a solid-form of a compound that crystallizes with one or more molecules of solvent trapped inside. A few examples of solvents that can be used to create solvates, such as pharmaceutically acceptable solvates, include, but are certainly not limited to, water, methanol, ethanol, isopropanol, butanol, C1-C6 alcohols in general (and optionally substituted), tetrahydrofuran, acetone, ethylene glycol, propylene glycol, acetic acid, formic acid, water, and solvent mixtures thereof. Other such biocompatible solvents which may aid in making a pharmaceutically acceptable solvate are well known in the art and applicable to the present disclosure. Additionally, various organic and inorganic acids and bases can be added or even used alone as the solvent to create a desired solvate. Such acids and bases are known in the art. When the solvent is water, the solvate can be referred to as a hydrate. Further, by being left in the atmosphere or recrystallized, the compounds of the present disclosure may absorb moisture, may include one or more molecules of water in the formed crystal, and thus become a hydrate. Even when such hydrates are formed, they are included in the term “solvate”.

The nitrogen oxide compound that is administered along with the nitrodilator can be one or more metabolites of NO. These metabolites of NO can be categorized, based on structural characteristics, into classes that include nitrogen oxoanions such as nitrite (NO₂⁻) and nitrate (NO₃⁻), S-nitrosothiols (SNOs), iron nitrosyl species (FeNOs) such as nitrosyl-heme (heme-NO) and dinitrosyl iron complexes (DNICs; non-heme-iron nitrosyl species), nitrated lipids, and NO sequestered in the hydrophobic regions of proteins. Importantly, some members of each of these categories of NO metabolites are stable enough to circulate systemically and also have NO-like bioactivity at physiological concentrations. These NO metabolites mediate their effects by protecting the NO from irreversible metabolism during its transit in the circulation and then releasing the same NO moiety at its distant site of action. As such, these compounds are often referred to as “NO storage” forms, or reservoirs of NO vasoactivity. Due to their varying chemical structures, the classes of NO metabolites possess a wide range of molecular weights, stabilities, and membrane permeabilities. However, despite these differences, this diverse group of these NO metabolites, which includes many SNOs and DNICs, demonstrate remarkably similar vasodilatory activity at potencies comparable to that of free NO itself. These compounds are collectively referred to as ‘nitrodilators’ based on five functional criteria (Table 1). First, like NO, all nitrodilators vasodilate via either direct or indirect activation of soluble guanylate cyclase (sGC) and the canonical cGMP vasodilatory pathway. Second, the nitrodilators have an initial vasodilatory potency that is comparable to or even greater than that of free NO itself. Third, different from the transient vasodilatory effect of free NO on isolated arteries, nitrodilators produce a vasodilatory response that is markedly more sustained. Fourth, they are relatively stable, and either do not release measurable free NO or they release it so slowly that their vasodilatory effects cannot be explained by virtue of free NO release. And finally, nitrodilators have a membrane permeability that is less than that of NO itself. This last characteristic is true of almost any molecule, given NO is a small, non-polar, uncharged molecule that is 3- to 4-fold more soluble in lipids than in aqueous solutions. However, it is important to highlight this characteristic of nitrodilators as it emphasizes the fact that it is not possible for release of the NO moiety from the nitrodilators to achieve the intracellular free NO concentrations that would be necessary to explain their ability to activate sGC at potencies comparable to NO itself. Indeed, the latter four characteristics of nitrodilators challenge the commonly held notion that they vasodilate simply by releasing an NO moiety which activates sGC. This disclosure capitalizes on an alternative explanation for the source of NO involved in nitrodilator-mediated sGC activation.

TABLE 1
Comparison of the chemistry and biological
properties of NO and various nitrodilators.

| | sGC- | Dilatory | Sustainability | Membrane | Ability to |
|---|---|---|---|---|---|
| Compounds | dependence | potency | of dilation | permeability | release NO |
| NO (authentic | + | + | − | ++ | ++ |
| NO donor) | | | | | |
| L-cysNO | + | + | + | + | + |
| D-cysNO | + | + | + | − | + |
| GSNO | + | + | + | − | − |
| Heme-NO | + | + | + | − | − |
| Glut-MDNIC | + | + | + | − | − |
| Glut-BDNIC | + | + | + | − | − |
| NTG | + | + | + | + | − |

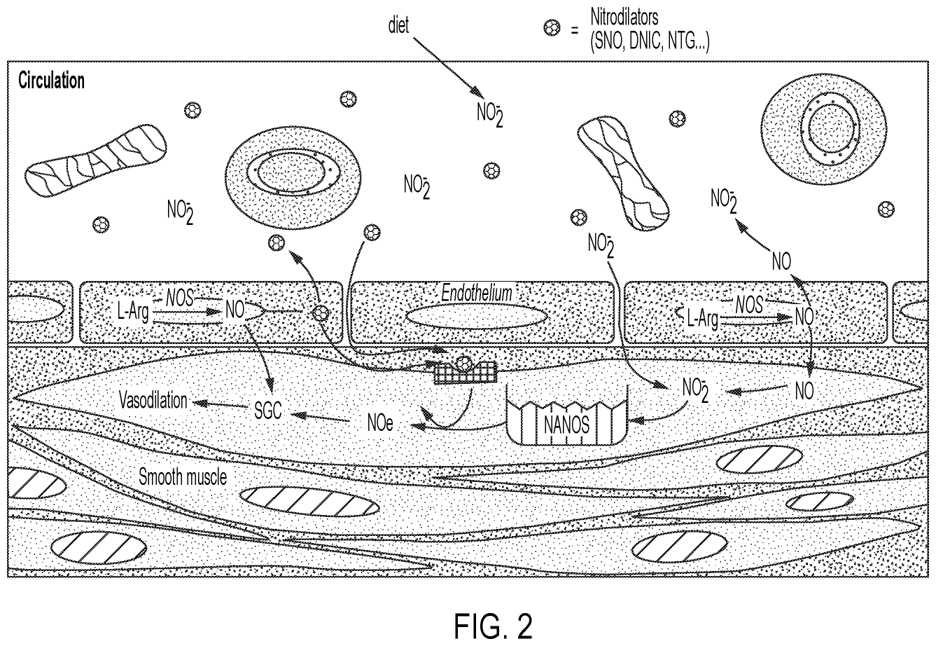

Vasodilation by nitrodilators involves the release of an NO-moiety from a store within the vascular smooth muscle cell (FIG. 2), in some respects similar to the activation of NO-mediated vasodilation in response to exposure to light. Nitrodilators cause vasodilation by an uncharacterized pathway, upstream of sGC activation, involving the release of an NO equivalent from a preformed store within the vascular smooth muscle cell, which is referred to as the nitrodilator-activated NO store (NANOS). NO synthetized by NO synthase in the endothelium is thought to diffuse randomly into the neighboring vascular smooth muscle where it can bind to the heme of sGC, resulting in cGMP-dependent vasodilation. It can also react in the blood and tissues to form various stable yet bioactive metabolites, with nitrite, SNOs, and DNICs being three most prominent.

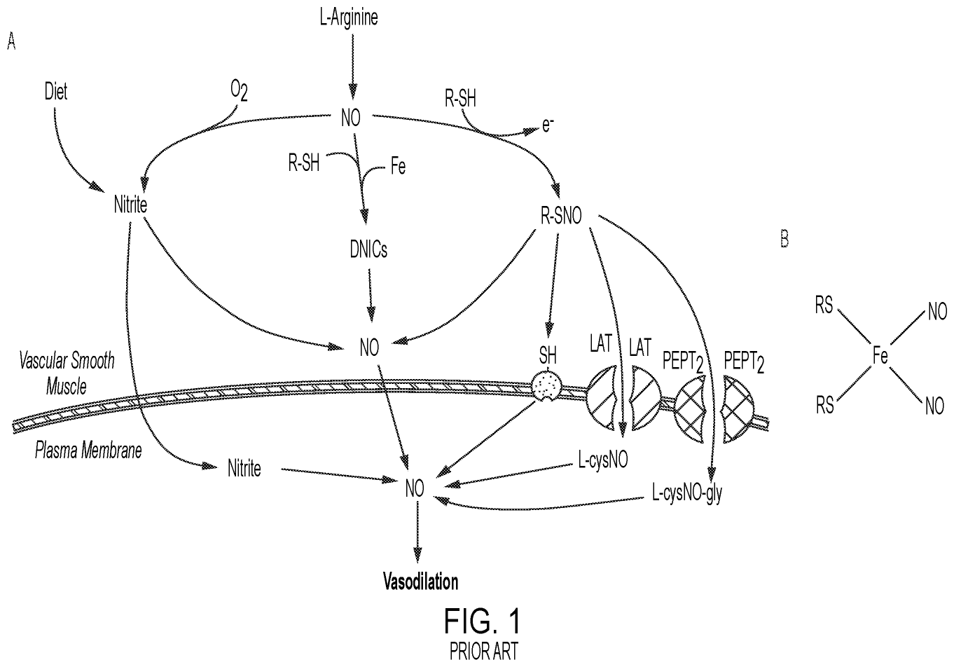

FIG. 1A (Prior Art) summarizes the current commonly held understanding of their production pathways and vasodilatory mechanisms. FIG. 1A illustrates the production pathways and vasodilatory mechanisms of three prominent NO storage forms: nitrite, DNICs, and SNOs. All three have been proposed to mediate vasodilation via various routes of conversion to free NO. DNICs and SNOs, most of which are membrane impermeable, have been proposed to release free NO outside the vascular smooth muscle cell, with the exception of possible selective transport of SNOs such as S-nitrosocysteine (L-cysNO) or L-cysNO-glycine (L-cysNO-gly) across the plasma membrane via the L-type amino acid transporter (LAT) or dipeptide transporters (PEPT2), respectively. Handoff of NO from extracellular SNOs to membrane thiols via transnitrosylation with subsequent transport of the NO to the cytoplasm has also been proposed.

Until the mid-1990s, nitrite was considered to be inert at physiological concentrations, and simply a byproduct of NO metabolism that is excreted in the urine. However, it is increasingly apparent that although nitrite is orders of magnitude less vasoactive than NO, it can serve as a source of NO bioactivity. A growing body of literature reports that increasing circulating nitrite concentrations by dietary intake results in decreased systemic vascular resistance and improved exercise performance. One proposed mechanism of nitrite's vasoactivity is based on the ability of deoxyhemoglobin to reduce nitrite to NO. By this reaction, the abundance of deoxyhemoglobin in hypoxic tissues would facilitate the conversion of nitrite into NO, leading to vasodilation that would serve to restore adequate O₂ delivery to hypoxic tissues. Consistent with this understanding, it has been demonstrated that the vasodilatory effects of nitrite are potentiated under hypoxic conditions. However, since erythrocyte hemoglobin scavenges free NO at rates roughly one million-fold faster than the rate at which deoxyhemoglobin produces NO from nitrite, how NO produced by this reaction escapes the red blood cells to reach the vessel wall remains unclear. Alternatively, a number of experiments present evidence that the vasodilatory effects of nitrite are not facilitated by deoxyhemoglobin. For instance, infusion of nitrite into either the relatively deoxygenated blood of the pulmonary artery, or of the femoral artery during systemic hypoxia, does not result in a decrease in pulmonary or femoral vascular resistance to flow until intravascular nitrite concentrations are orders of magnitude above the physiologic range. Likewise, although fetal hemoglobin reduces nitrite to NO twice as fast as adult hemoglobin does, and normal fetal oxyhemoglobin saturations are at a level that favors the reduction of nitrite to NO, infusion of nitrite into chronically instrumented fetal sheep does not result in cerebral vasodilation until plasma nitrite concentrations reach>100-fold greater than the physiological range.

As an alternative to the hemoglobin reductase hypothesis, vasodilating effects of nitrite appear to be derived from its direct action in the vascular wall. As indicated by data herein, the vasodilatory effects of treatment with nitrite are found to persist even after plasma nitrite levels have returned to baseline. The mechanism for these effects remains unclear, although it may involve the reduction of nitrite to NO, or some NO-adduct, within the vascular smooth muscle cell by a number of different proposed pathways.

Intriguingly, measurements of nitrite concentrations also indicate it may play a special role in vascular smooth muscle. For example, while concentrations measured in most tissues are well below 1 μM, including the blood, concentrations in the walls of an animal aorta range from 5 to 25 μM. The presence of such high concentrations is consistent with the existence of the NANOS in the vessel wall. the concentrations measured in the aorta are not necessarily the same as those found in the downstream resistance vessels where vascular tone is more important in controlling blood pressure.

S-nitrosothiols, a category of NO adduct with potent vasodilatory effects, are formed by the addition of NO to the thiols of molecules ranging in size from small molecules such as thionitrous acid (HSNO) and nitrosoglutathione (GSNO) to high molecular weight compounds such as the nitrosylated cysteine residues of large proteins such as hemoglobin and albumin. In addition, SNOs can be produced by the ‘handoff’ of an NO moiety from one SNO to another thiol, a reaction called transnitrosylation (R₁—SNO+R₂—SH⇔R₁—SH+R₂—SNO, where R₁ and R₂ are different molecules). As a result of transnitrosylation, the various SNOs in plasma exist at equilibrium with each other, with the more stable high molecular weight (HMW) forms such as albumin-SNO predominating. For example, intravascularly-infused low molecular weight (LMW) SNOs such as S-nitroso-cysteine (cysNO) or S-nitroso-glutathione (GSNO) are converted to HMW plasma SNOs within one systemic circulatory transit time. Advancing the understanding that SNOs may be a circulating form of NO bioactivity, Stamler et al proposed that a cysteine thiol on the beta subunits (β-93) of hemoglobin constitutes an important carrier of NO in the blood. Albeit not without challenge, there is evidence that the NO moiety of hemoglobin-SNO can be exported out of erythrocytes in the form GSNO via a process that is facilitated by the R-to-T shift in conformation that occurs upon deoxygenation. As discussed in the next section, GSNO and other membrane-impermeable SNOs are potent vasodilators. What is still not understood is how the exported GSNO, which is membrane impermeable and does not release free NO, would be able to cause vasodilation.

The mechanisms underlying SNO-mediated vasodilation have puzzled investigators in the field for over four decades. Like NO, SNOs cause vasodilation primarily via activation of sGC, although they are also capable of signaling via transnitrosylation of proteins, resulting in posttranslational modification of protein function. However, the mechanism by which extracellular SNOs activate cytosolic sGC is still not clear. Due to their different thiol ligands, SNOs can have widely-varying stabilities and membrane permeabilities (Table 1). For instance, L-cysNO and its stereoisomer D-cysNO are 10-fold less stable than GSNO and thus more likely to release NO, although L-cysNO releases merely<10% of its NO moiety over a duration of one hour in the presence of metal chelator in PBS. On the other hand, Hogg et al demonstrated that L-cysNO can be taken up into cells via the L-type amino acid transporter (LAT), whereas D-cysNO and GSNO (200-fold less membrane permeable than L-cysNO) are largely membrane-impermeable. Based on these properties, one might expect the vasodilatory potency of these three compounds to differ greatly, with the L-cysNO being the most potent and GSNO being the least potent. However, despite these different rates of decomposition, stereo conformation, and membrane permeability, all three of these SNOs have vasodilatory potencies comparable to NO itself. In other words, although a majority of the NO moiety of GSNO remains outside the cell, GSNO is able to activate sGC with potency equal to that of L-cysNO and even free NO itself, both of which enter the cell much more rapidly. Although multiple mechanisms have been proposed to explain this phenomenon (FIG. 1A), no consensus of support has emerged for any one of these pathways.

Dinitrosyl iron complexes are a class of FeNOs that contain complexes of NO with ferrous non-heme-iron (FIG. 1B) and usually thiol ligands, although other ligands have also been described. FIG. 1B is a representation of the structure of a mononuclear dinitrosyl iron complex. These compounds contain one (mononuclear) or two (binuclear) Fe(NO) 2 nuclei (MDNICs or BDNICs, respectively). Similar to SNOs, DNICs are capable of transferring their Fe(NO) 2 nuclei to thiols on other molecules, resulting in a wide variety of DNICs with varying molecular weights, structures and stabilities. DNICs have been found to vasodilate isolated, pre-constricted arteries at submicromolar concentrations—an effect that is blocked by inhibition of sGC. For example, glutathione-liganded DNICs have a vasodilatory EC₅₀ on the order of 1 μM, comparable to that of free NO itself. As with SNOs, the comparable vasodilatory potencies of glutathione-liganded DNIC and free NO raises a mechanistic conundrum regarding how DNIC that is membrane-impermeable and stable activate intracellular sGC just as potently as free NO itself, and how DNICs and SNOs produce a vasodilatory response that is markedly more sustained than that of free NO.

While DNICs are clearly potent vasodilators with pharmacologic potential, their physiologic relevance remains unclear due to a lack of assay methodologies with adequate selectivity and sensitivity to measure DNICs in blood and tissues. The gold standard method, electron paramagnetic spectroscopy (EPR), is capable of distinguishing DNICs from other NO metabolites. However, the lower limit of detection of EPR is at or above the vasoactive concentrations of DNICs, and thus EPR cannot definitively determine whether DNICs are present at vasoactive concentrations. Ozone-based chemiluminescence offers the ability to detect DNICs at concentrations that are within or below the vasoactive range. However, this method requires sample handling and pre-processing that may result in the degradation of DNICs to form other NO metabolites before they reach the assay instrument. In spite of these interchanges, cell culture experiments with both exogenous and endogenous NO sources suggest DNICs are the most abundant intracellular NO adduct. DNICs are the “working form” of NO in cells and tissues, with high molecular weight DNICs represent a predominant reservoir of intracellular NO bioactivity while the less stable LMW DNICs transfer the NO bioactivity to heme- or thiol-containing targets.

As outlined earlier, the existence of a light-sensitive NO store in the vascular muscle is well established. For example, precontracted endothelium-denuded arteries are relaxed by exposure to light particularly UV light, and this photo-relaxation is due to NO-mediated activation of sGC, despite the absence of endothelial NO production. Moncada et al demonstrated that the photo activated NO store can be depleted by repeated exposure of the arteries to light, and that it can also be gradually replenished when endothelium-derived NO production is preserved or when endothelium-denuded arteries are incubated with an NO donor. The activation mechanism for the light-sensitive NO store is not well understood. Earlier reports favored UV light irradiation as the activator of the NO store. However, more recent work found far red, near infrared, and visible light are also capable of activating photo relaxation. In fact, the nature of the vasodilatory response varies with respect to the wavelength of light used for stimulation. For example, visible light causes a transient and reversible relaxation that is endothelium-dependent, whereas the relaxation caused by UV light includes an initial transient response superimposed on a sustained one that is endothelium-independent. This evidence has led to proposals of two or more light-sensitive NO stores that are distinct in chemical nature, one in the endothelium and the other in the vascular smooth muscle cells.

In addition to light, LMW thiols at low millimolar levels, far in excess of physiological levels, can also activate an NO store and mediate endothelium-independent vasodilation, although it remains to be determined whether this thiol-sensitive NO store is identical to the light-sensitive one. Muller et al demonstrated that membrane-permeable N-acetyl-L-cysteine (NAC) relaxes rat aortas that were pretreated with LPS and L-arginine to form a putative intracellular NO store, but not those pretreated with LPS or L-arginine alone, or those with LPS, L-arginine, and L-NAME. It has been proposed that LMW thiols facilitate the dissociation of NO from intracellular NO storage forms to cause vasodilation.

The chemical nature of the light-sensitive NO store has only been partially characterized. SNOs, heme-bound NO, and DNICs are all capable of photolytic release of NO under irradiation by UV, red, and infrared light. In contrast, the photolytic lability of nitrite is relatively low, such that unphysiologically high intracellular concentrations (>1 mM) of nitrite would be required for photo relaxation.

Several lines of evidence suggest a role for SNOs as an intracellular NO store. For example, exposure of isolated arteries to compounds that deplete SNOs attenuates both light- and thiol-mediated relaxation. Conversely, incubating vessels with membrane-permeable SNOs to increase concentrations of NO adducts in the vessel wall augments thiol-mediated relaxation. In addition, exposure of arteries to red light stimulates the release of a vasodilator with characteristics of SNOs from the vessel, highlighting the close relation of SNOs to the intracellular NO store.

There is also evidence supporting a role for DNICs as an intracellular NO store. Pre-incubation of arteries with LPS and L-arginine in combination, or with an NO donor leads to an increased intracellular store of DNICs with protein ligands that can be mobilized by membrane-permeable LMW thiols to release NO in the form of LMW thiol-liganded DNICs, leading to vasodilation. However, because SNOs, DNICs, and other NO adducts are interconvertible and are likely to exist in equilibrium, it is possible that they all play some role in the function of the intracellular NO store.

Although a vast majority of the vascular smooth muscle in the body is shielded from the amount of light that would be necessary to activate NO release from the photosensitive NO stores, both light- and thiol-sensitive NO stores in the vasculature have been associated indirectly with cardiovascular function and pathology, suggesting they participate in vascular smooth muscle function even in the absence of light. For example, in young, spontaneously hypertensive rats, the function of the thiol-sensitive NO store decreases in parallel with the development of endothelial dysfunction and hypertension. Conditioning with mild intermediate hypoxia, on the other hand, enhances the function of the store and also prevents endothelial dysfunction and hypertension. In humans, Feelisch et al. suggested that UV light modulates systemic NO bioavailability by mobilizing the NO store in the skin, and that this might contribute to the latitudinal and seasonal variation of blood pressure and cardiovascular diseases.

Inventors recognized that nitrodilators utilize the intracellular NO store, herein the “NANOS”, to effect vasodilation based on experiments intended to determine the mechanism underlying SNO-mediated vasodilation. Previous hypotheses were based on the assumption that these NO adducts release their NO moiety, which then directly activates sGC (FIG. 1A). Many pathways by which the NO of the extracellular SNO might reach the intracellular sGC have been proposed. However, experiments herein indicate none of these pathways play a key role. Instead, these results raised the alternative possibility that SNO causes vasodilation via mobilization of an NO moiety from a source other than the extracellular SNO itself—essentially a preexisting intracellular NO store reminiscent of the light-sensitive one.

As shown schematically in FIG. 2, the NANOS model indicates that a preformed intracellular NO store is mobilized by extracellular nitrodilators to mediate vasodilation. Inhibition of endogenous NOS and/or repeated stimulation by these nitrodilators attenuates the vasodilatory response to subsequent nitrodilator exposure via depletion of the NANOS. Furthermore, incubation of arteries with compounds capable of contributing to the store potentiates nitrodilator-mediated vasodilation.

One expectation consistent with the concept of a NANOS would be that its contents can be depleted, and that its function would diminish accordingly, as has been demonstrated repeatedly for the light-sensitive NO store. Indeed, consistent with depletion, isolated vessels lose ˜50% of their initial vasodilatory response to GSNO after repeated exposures to GSNO, despite retaining a full response to NO itself. This finding is consistent with a use-dependent loss of GSNO-mediated vasodilation that results from attenuation of a pathway component that lies upstream of sGC activation. The initial exposure of arteries to GSNO triggers mobilization of the NANOS, which becomes depleted, leads to an attenuated response to subsequent GSNO exposures.

Based on the understanding that endogenous eNOS activity is necessary to constitutively maintain the NANOS, treatment with L-NAME was evaluated for its ability to attenuate vasodilatory responses to GSNO in intact rats and sheep. In both cases, animals treated with L-NAME had a greatly diminished or even absent femoral vasodilation in response to GSNO, D-cysNO, and L-cysNO. Interpreted in the context of the NANOS model, these results suggest that inhibition of endogenous NO production by NOS leads to depletion of the NANOS-dependent vasodilation caused by SNOs.

In further accordance with a NANOS model, fortifying the store should potentiate vasodilatory responses to nitrodilators. To increase the store experimentally, isolated sheep femoral arteries were pre-exposed to 1 μM nitrite, a concentration two-to-three orders of magnitude below which nitrite itself causes vasodilation. Such treatment was found to potentiate vasodilatory responses to GSNO. In addition, the attenuation of vasodilatory response to GSNO that occurs in isolated arteries after repeated exposure to GSNO to deplete the NANOS can be reversed by subsequent incubation of the arteries with 1 μM nitrite for 30 min. Likewise, intravenous infusion of sub-vasodilatory concentrations of nitrite to intact rats and sheep pre-treated with L-NAME led to potentiated vasodilatory responses to subsequent infusions of SNOs in the femoral arteries that are otherwise not responsive to SNOs. Interpreted in the context of the NANOS model, these findings suggest that nitrite can replenish the NO store after it has been depleted by inhibition of endogenous NOS activity.

A natural example of a reduced NO store is found in newborn humans and lambs. In both species, their plasma nitrite concentration decreases by more than 50% within minutes after birth to levels even lower than those observed following NOS inhibition. During this postnatal period, treatment of newborn lambs with low micromolar concentrations of intravenous nitrite is found to potentiate GSNO-mediated vasodilation. This finding is again consistent with the understanding that nitrite serves to replenish the NANOS.

Surprisingly, under some conditions L-NAME itself can contribute to the NANOS. In contrast to acute administration of L-NAME, pretreatment of rats with L-NAME for four days potentiated the mesenteric vasodilatory response to nitrodilators but not to NO itself, a finding that suggests upregulation of a pathway component that lies upstream of sGC activation, reminiscent of the enlargement of the NO store. L-NAME itself contributed directly to the NANOS in mesenteric arteries, and L-NAME had increased the concentration of NO-containing compounds in the mesenteric arterial wall. In addition, in vitro and in vivo application of L-NAME that had an isotopically labeled nitro group (R-15NO₂) demonstrated that metabolic conversion of L-NAME releases NO from its nitro group.

As further evidence that the potentiating effects of L-NAME are due to its release of NO but not to NOS inhibition, its stereoisomer D-NAME, which does not inhibit NOS but does release NO from its nitro group, also potentiated nitrodilators-mediated vasodilation. Furthermore, the potentiation was not observed after treatment with L-NMMA, a NOS inhibitor that does not contain a nitro group. In addition, the potentiating effects of L-NAME were augmented in the presence of oxidative stress, which was found to facilitate the release of NO from the nitro group. Thus, there is evidence that chronic treatment with L-NAME can potentiate the mesenteric vasodilation of nitrodilators via contribution of its nitro group to the NO store.

Nitrite likely contributes to the NO store via reduction into NO, but the underlying mechanism remains unclear. A number of different metalloproteins such as hemoglobin (Hb), myoglobin, xanthine oxidase, cytochrome C oxidase, and eNOS have been proposed to be involved in the reduction of nitrite to NO. However, in most cases, O₂ competes with nitrite for metal active sites on these proteins so effectively that their ability to reduce nitrite to NO is largely inhibited under physiological O₂ tensions. Furthermore, the oxygenated forms of many of these metalloproteins avidly scavenge NO, converting it to nitrate. Thus, the importance of these pathways of conversion of nitrite to NO under physiological conditions, particularly in the wall of resistance vessels where PO₂s are near arterial levels, is questionable.

At low pH, nitrite can also be converted to NO via disproportionation. However, the pKa for nitrite is 3.3, and thus the contribution of this pathway to nitrite reduction would also be very limited at physiological pH. Oxidative stress facilitates the NO release and NANOS-augmenting properties of the nitro group of L-NAME.

Despite representing a wide range of chemical reactivities, molecular sizes, and stoichiometries, DNICs and nitroglycerin appear to cause vasodilation in a manner similar to SNOs. These NO-moiety containing compounds cause vasodilation with potencies comparable to that of NO itself, and by activation of sGC, but the mechanisms by which they activate sGC remain unclear. Although these compounds are often considered to be NO donors, most SNOs and DNICs differ from authentic NO donors, such as NONOates, in that they do not readily release free NO and also because they cause a sustained relaxation in isolated vessels unlike the more transient vasodilation elicited by NO donors. Likewise, NTG has been thought to activate sGC by releasing the NO moiety from its nitrate group via the action of enzymes such as aldehyde dehydrogenase-2. However, this conversion produces nitrite rather than NO, especially at therapeutic NTG concentrations (<1 μM). Meanwhile, with an EC₅₀ of <0.1 μM, the vasodilatory sensitivity of NTG is approximately one order of magnitude greater than that of NO per se when tested in isolated arteries. These similarities led us to reclassify these NO-moiety-containing vasodilators as nitrodilators, a designation that does not include authentic NO donors like NONOates. This term also serves to categorize these compounds based on their common role as stimulators of the NANOS, as suggested above for SNOs. Consistent with this classification, vasodilation of the mesenteric vasculature of rats by GSNO, glut-BDNIC and NTG are all potentiated by chronic pretreatment with L-NAME and D-NAME, which have a nitro group that may contribute to the NO store, but not by pretreatment with L-NMMA or L-arginine, both of which lack a nitro group. In addition, the potentiation effects are all augmented under oxidative stress, which may facilitate the contribution of nitro group to the NO store via the release of NO. This evidence further justifies the reclassification of SNOs, DNICs, and NTG as nitrodilators, and supports their role as stimulators of the intracellular NO store.

NTG causes tolerance, a loss of responsiveness after repeated use. Interestingly, NTG tolerance is often accompanied with a cross-tolerance to other nitrodilators and even endothelium-dependent vasodilators. Although cross-tolerance also occurs with some so-called NO donors such as sodium nitroprusside, these chemicals actually fall into the category of nitrodilators in that they do not release significant amounts of free NO. For authentic donors of NO such as DEA-NONOate, cross-tolerance is not observed. In addition, the NTG tolerance can be gradually reversed by a drug-free interval. These features of tolerance that develops to nitrodilators such as NTG are consistent with the depletion of an intravascular NANOS.

Under the NANOS working model, the endothelium provides a constitutive source of NO and nitrite for integration into the NANOS of the neighboring vascular smooth muscle. Accordingly, prolonged inhibition of endothelial NOS results in depletion of the NANOS and attenuation of nitrodilator-mediated vasodilation. There is also evidence that the endothelium itself may release SNOs and DNICs in addition to free NO. Significant aspects of work in this area are in keeping with the NANOS model. For example, vasodilation in response to bradykinin decreases upon repeated use, and this decrease occurs more rapidly in the presence of NOS inhibition. The use-dependent loss of vasodilation is also observed for acetylcholine-mediated vasodilation in the presence of NOS inhibition, despite no change in the vasodilatory response to NO donors. Furthermore, recent work demonstrates that endothelium-dependent flow-mediated vasodilation in humans is improved by dietary nitrite and nitrate, which may contribute to the intracellular NO store. Together, these facts raise the possibility that endothelium-dependent vasodilation may be at least partly dependent upon the NANOS.

NANOS involves contributors, nitrodilators, trans-membrane signaling to activate the store, the constituents of the store itself, NO equivalents, and mechanisms for the uptake, retention, and release of NO bioactivity by the store.

As shown in FIG. 2, the direct contributors to the store include NO produced by endothelial NOS, nitrite, and nitro-group-containing compounds. Nitrodilators, including SNOs, DNICs, and NTG, are shown as well-established vasodilators that would stimulate the NO store. Moreover, the endothelium is shown to act as a source of nitrodilators, in addition to release of free NO. A trans-membrane signaling mechanism is included to account for cross-membrane vasodilatory signaling of nitrodilators, which are membrane-impermeable and stable. Such a membrane receptor/transporter would seem likely to depend on recognition of the NO moiety because it is common to all the nitrodilators.

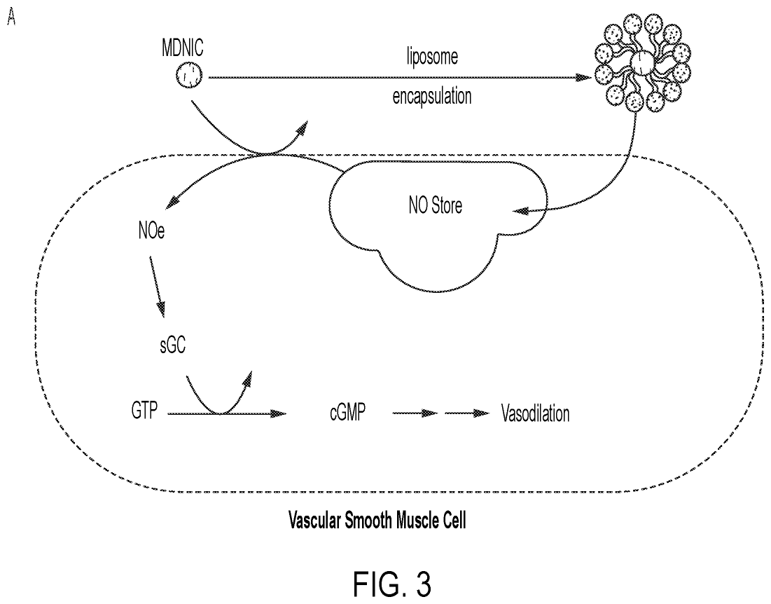

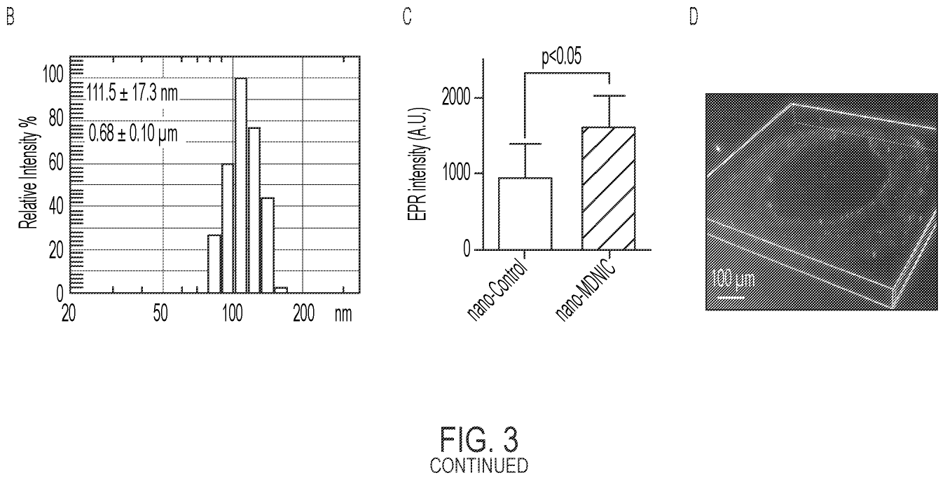

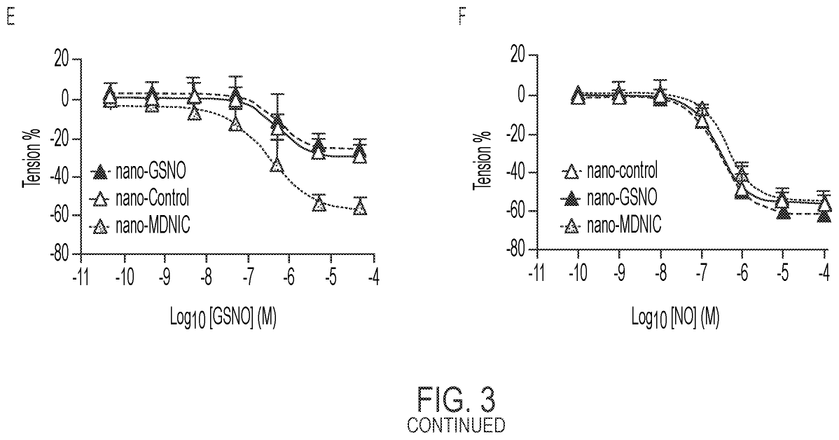

The NANOS model is reminiscent of the intracellular calcium store, where actual calcium cations constitute the store and mechanisms exist for their uptake, retention, and intracellular release to facilitate vasoconstriction. However, the molecular components of NANOS and the identity of the components in the light- and thiol-sensitive NO stores are not fully known. Because NO is a highly reactive and freely diffusive gas, it is less likely to be stored and regulated as such. Rather, the NO store is likely to be a more stable NO adduct such as SNOs and DNICs. Taking advantage of a cell-based model that is amenable to surrogate genetic approaches regulating cellular GSNO level via overexpression or knockdown of GSNO reductase, intracellular GSNO was evaluated for its role in mediating the cGMP-dependent pathway. To test if the intracellular NO store is related to GSNO, GSNO encapsulated within nanoliposomes were used to preload GSNO into the arterial wall while avoiding potential mobilization of the NO store through extracellular stimulation. Preloading the arteries with liposomal GSNO did not reverse the use-dependent loss of vasodilatory response to GSNO, suggesting that GSNO is an unlikely candidate for the NO store. On the other hand, parallel experiments with glutathione-liganded MDNIC encapsulated in nanoliposomes raised DNIC levels in the artery and reversed the use-dependent tolerance to GSNO-mediated vasodilation without altering NO-mediated vasodilation (FIGS. 3A-3F). These findings support intracellular DNICs as a candidate for the NANOS.

FIGS. 3A-3F demonstrate MDNICs as a candidate of the NANOS (n=5). FIG. 3A is an illustration of the contribution of the NANOS by nanoliposomes containing MDNIC. FIG. 3B is a graphical representation of the diameter of nanoliposomes that enclose glut-MDNIC, with a mean concentration of 0.68 μM. FIG. 3C is a graphical representation of the electron paramagnetic resonance (EPR) intensity of the control and MDNIC-containing nanoliposomes. Incubation with MDNIC-containing nanoliposomes introduces MDNIC into the arteries as measured by electron paramagnetic resonance spectroscopy. FIG. 3D is a photographical representation of the uptake of nanoliposomes tagged with Texas Red into the arterial wall as evidenced by fluorescence image. FIG. 3E is a graphical representation of the response of arteries following incubation with nanoliposomes containing MDNIC, demonstrating a reversal of loss of vasodilatory response that occurs following repeated exposure to GSNO, while nanoliposomes encapsulated with GSNO or buffer (empty) do not. FIG. 3F is a graphical representation of the response of arteries following incubation with nanoliposomes containing MDNIC, demonstrating that the incubation does not alter NO-mediated vasodilation for all three samples.

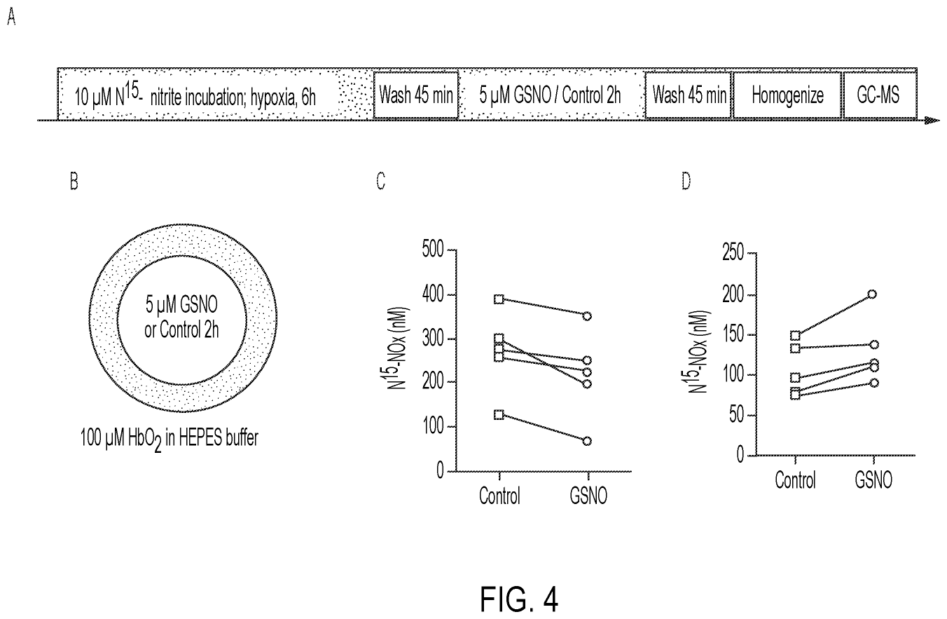

DNICs function as an intracellular NO store. NO can be stored as DNICs in ferritin, a cytosolic protein that stores and releases iron in controlled fashion. Nitrite, a NO store contributor, results in considerable cytosolic DNICs in experiments with isolated tissues and in whole animals. Incubation of cells with GSNO, a putative NANOS activator, stimulates mobilization of intracellular DNICs, a complicated process that involves glucose metabolism, iron release, glutathione efflux, and the cooperation of carrier and transporter of DNICs. In accordance with this, the general mobilization of arterial intracellular NO-containing species (NOx) upon extracellular stimulation of nitrodilator has recently been observed. As shown in FIGS. 4A-4D, incubation with GSNO resulted in an efflux of NOx from the arterial wall, providing physical evidence in agreement with the NANOS model. FIGS. 4A-4D demonstrate the mobilization of the NANOS. FIG. 4A is an illustration of the protocol. A sheep carotid artery (length 8 cm) was incubated with 10 μM N¹⁵-nitrite in DMEM under 10.5% O₂ for 6 h to load the vessel with N¹⁵-NOx (NO metabolites). FIG. 4B is an illustration of the prepared vessels. After extensive washes, the arteries were well sealed and incubated for 2 h with 3 ml 100 μM oxyhemoglobin in HEPES buffer on the extraluminal side and 5 μM GSNO or control (0.7 ml HEPES buffer containing 0.1 mM DTPA) on the luminal side. FIG. 4C is a graphical representation of the response of the arteries exposed to GSNO. The N¹⁵-NOx levels were lower (p=0.0206) in the homogenate of arteries exposed to GSNO. FIG. 4D is a graphical representation of the response of the arteries exposed to intraluminal GSNO. The N¹⁵-NOx in extraluminal buffer was higher (p=0.0400) in the arteries exposed to intraluminal GSNO. N¹⁵-NOx was measured by GC-MS. The results are consistent with intraluminal GSNO stimulating an efflux of NOx from the vascular smooth muscle as manifested by a decrease in intracellular NOx and an increase in extracellular NOx.

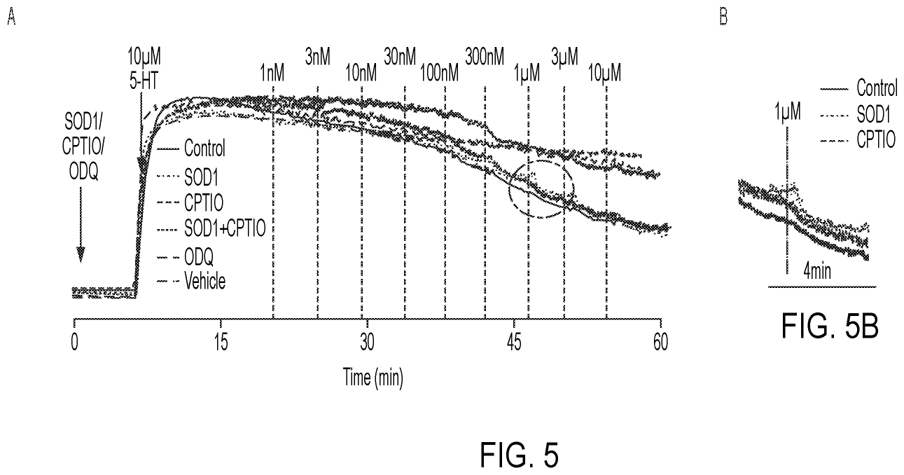

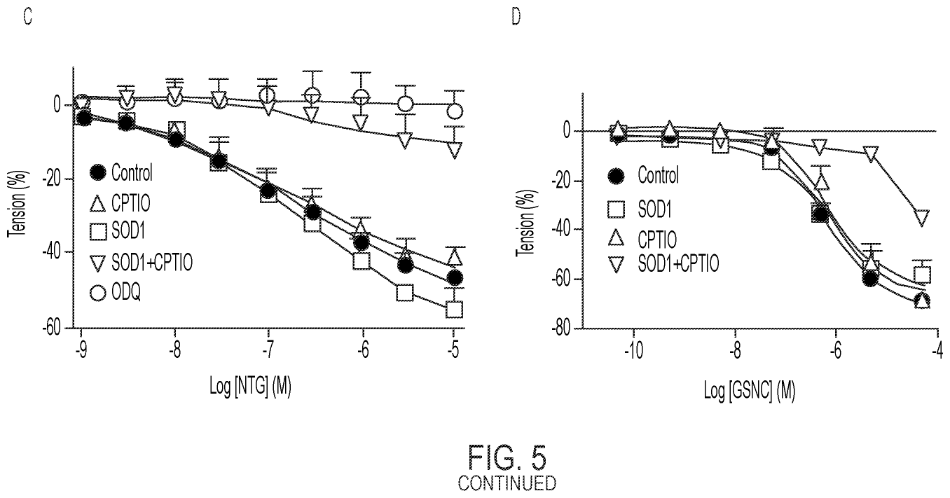

As discussed above, there are reasons to doubt whether NO itself is the mediator of sGC activation in the NANOS model. Rather, an equivalent of NO may be released by activation of the NANOS. Despite persisting controversy, HNO, the redox cousin of NO, has been suggested to be an alternative activator of sGC. The NO equivalent has HNO-like characteristics. As shown in FIGS. 5A-5D, CPTIO, a membrane-impermeable NO scavenger, does not affect NTG- or GSNO-mediated relaxation, whereas SOD1, which converts HNO into NO, enables inhibition of vasodilation by CPTIO. In addition, similar effects of SOD1 on CPTIO were also observed for glut-BDNIC and the HNO donor Angeli's salt. Finally, like SOD1, TEMPOL and CuCl, chemicals that convert HNO into NO but do not affect vasodilation per se, also enable the inhibitory effects of CPTIO. FIGS. 5A-5D demonstrate the HNO-like properties of the NO equivalent released from the NANOS. Isolated sheep mesenteric arteries were denuded of endothelium and contracted with 10 μM 5-HT (n=5). FIG. 5A presents examples of relaxation traces (one animal, six arterial strips) of NTG in the absence and presence of an sGC inhibitor (ODQ) (10 μM) or superoxide dismutase (SOD1) (1000 U/ml, converts HNO into NO, or the extracellular NO scavenger CPTIO (200 μM), or SOD1 and CPTIO together. All traces were normalized for comparison of extent of relaxation. FIG. 5B is a graphical representation of the amplification of the area in the red circle in ‘A’. FIG. 5C is a graphical representation of the effects of SOD1, CPTIO, SOD1+CPTIO, and ODQ on relaxation in response to Nitroglycerin (NTG). FIG. 5D is a graphical representation of the effects of SOD1, CPTIO, and SOD1+CPTIO on relaxation in response to nitrosoglutathione (GSNO). SOD1, via putative conversion of HNO to NO, enables the inhibitory effects of CPTIO on relaxation caused by NTG and GSNO.

As discussed above, the endothelium may play a role in the NANOS via the production of nitrodilators as stimulators and the production of NO and nitrite as contributors. This correlation adds another dimension to the connotation of endothelial function, one of the most valuable predictors of future cardiovascular events. Conversely, it also has significant physiological and pathological implications for the NANOS model. Consistent with this rationale, plasma levels of nitrite, which correlate to the NO store function, have been suggested to reflect the degree of endothelial function. Extrapolating from endothelial NOS, NO sources such as inducible and neuronal NOS and dietary nitrite and nitrate may also play a role in the NANOS model. Through their impacts on these NO sources, various physiological and pathological conditions could be related to the NANOS. It is worth noting that physiological and pathological conditions may not merely affect the size of the NANOS. As has been suggested for the thiol-sensitive NO store, the size, capacity, and efficiency of the NO store might all alter depending on the conditions.

The presence of a NANOS also provides opportunities for advancing medical treatments. Examples in which modulation of the NANOS can have therapeutic potential include treatment of a wide variety of hypertensive disorders, hypercoagulation, and improved exercise performance, where dietary nitrite has already proven effective. A specific example where increasing the NANOS could be of marked benefit is in avoiding tolerance to nitroglycerin, a century-old problem in treatment of angina pectoris.

The proposal of a NANOS participating in the regulation of vascular tone is a novel notion that challenges the fundamental paradigm of NO signaling. In contrast to the conventional thought of NO as a freely diffusive gas that signals without need for a membrane receptor, conceptually, the NANOS mediates a controlled mobilization of NO bioactivity upon extracellular stimulation. In addition, the NANOS model may also shed new light on understanding the signaling of different NO storage forms. The NANOS model provides a framework for the crosstalk between different NO species, such as that between nitrite and SNOs in vasodilation. This model not only provides potential explanations for the mechanisms by which nitrite and SNOs cause vasodilation, but also builds a conceptual bridge between evidence for the endocrine vasodilatory functions of nitrite and SNOs. Moreover, the synergistic actions of intracellular and extracellular NO species in vasodilation, that is the NANOS in the vessel wall and the circulating nitrodilators, adds new temporal and spatial aspects to the signaling of NO and its storage forms.

SNOs, DNICs, heme-NO, nitroprusside, NTG, and other nitrodilators, dilate vessels via activation of soluble guanylyl cyclase (sGC) in vascular smooth muscle cells. These nitrodilators have potent NO-mimetic vasoactivities despite not releasing requisite amounts of free NO. These nitrodilators activate sGC via a preformed NANOS found within the vascular smooth muscle cell. Other examples of the nitrodilators include, but are not limited to, isosorbide dinitrate, isosorbide mononitrate, and nitrosyl complexes with metals or metalloproteins with metals other than iron. The effects of nitrite on reverting NTG tolerance of coronary arteries have been confirmed both in vivo and in vitro. The potentiation effects of nitro-group containing chemicals on vasodilation by nitrodilators including NTG have been confirmed both in vivo and in vitro.

Both exogenous and endogenous NO is readily metabolized into nitrodilators such as SNOs, DNICs, and heme-NO. For instance, it has been demonstrated that intravascularly applied NO generates SNOs in the circulation. Endothelium-derived relaxing factor (EDRF) contains not only NO itself but also NO metabolites as its components. Even as a minor component, these NO metabolites, many of which are nitrodilators, are of major importance for cardiovascular health. Therefore, the nitrodilator-NANOS signaling axis also plays an important role in the actions of both exogenous and endogenous NO. A perturbation that contributes to the NANOS may be applied in addition to treatment that relies on delivery of exogenous NO or production of endogenous NO to enhance the treatment. In an embodiment, inhalation of NO results in production of SNOs that activate the NANOS. Via its contribution to the NANOS, nitrite can potentiate the NANOS activation mediated by the SNOs generated during NO inhalation. Likewise, a perturbation that facilitates the generation of nitrodilators from NO treatment can enhance the treatment. In an embodiment, inhalation of NO together with nebulized thiols, such as L-cysteine, results in production of L-cysNO that signals via activation of the NANOS.

Embodiments include methods of treating a vascular or a respiratory condition in a human subject by administering to the subject a therapeutically effective amount of a nitrogen oxide compound to contribute to the NANOS along with a source of exogenous NO. In certain embodiments, the nitrogen oxide compound is nitrite. In certain embodiments, the exogenous NO is inhalation of NO. Normal plasma nitrite is about less than 0.5 μM. Administration of the nitrogen oxide compound along with a source of exogenous NO results in an increased plasma nitrite of about 1 μM to about 10 μM. Circulating nitrite levels are usually measured by one of several techniques known in the art, such as ozone-based chemiluminescence.

Embodiments include methods of treating a vascular or a respiratory condition in a human subject by administering to the subject a therapeutically effective amount of thiol, which facilitates generation of SNOs to activate the NANOs, along with a source of exogenous NO. In certain embodiments, the exogenous NO is inhalation of NO. In certain embodiments, the thiol is cysteine or glutathione. In certain embodiments, the delivery of thiol is inhalation of nebulized thiol.

Embodiments include methods of treating a vascular or a respiratory condition in a human subject by administering to the subject a therapeutically effective amount of a nitrogen oxide compound along with a source of endogenous NO. Administration of the nitrogen oxide compound along with a source of endogenous NO results in an increase in plasma nitrite to about 1 μM to about 10 μM. Circulating nitrite levels are usually measured by one of several techniques known in the art, such as ozone-based chemiluminescence. In certain embodiments, endogenous NO is administered as precursors to NO production, such as the amino acids L-arginine or L-citrulline or combinations thereof.

In certain embodiments, the combined application of a nitrodilator and a nitrogen oxide compound is affected by a perturbation in the sGC-cGMP pathway. Vasodilation mediated by the nitrodilator and/or nitrogen oxide compound can be further facilitated by agents that enhance the sGC-cGMP pathway. Such agents include sGC stimulators and activators, cGMP-phosphodiesterase inhibitors, and others. The sGC stimulators directly activate reduced-heme-containing sGC that generates cGMP to cause vasodilation. The sGC activators activate sGC when the heme is oxidized or the heme moiety is missing. A sGC stimulator can be riociguat or vericiguat. The sGC activator can be runcaciguat, cinaciguat, ataciguat, or kynurenine. The cGMP-phosphodiesterase inhibitors, such as sildenafil, vardenafil, and tadalafil, stabilize cGMP via preventing its degradation by phosphodiesterase-5. In an embodiment, riociguat, sildenafil, or a combination of both is administered in addition to nitrogen oxides and/or nitrodilators, such as nitrite, nitroglycerin, or both, thereby maximizing the vasodilatory effects of activation of the NANOS. Certain embodiments include methods of treating a vascular condition or a respiratory condition in a human subject by administering a therapeutically effective amount of a nitrogen oxide compound and a cGMP-phosphodiesterase inhibitor. For example, a method of treating erectile dysfunction in a subject can include administering a therapeutically effective amount of a nitrogen oxide compound, such as a nitrate-group containing compound or a nitrite compound and a cGMP-phosphodiesterase inhibitor, such as sildenafil, vardenafil, or tadalafil. The potentiation effects of riociguat and sildenafil on vasodilation by nitrodilators including GSNO have been confirmed in vitro.

Embodiments include methods of treating a vascular condition or a respiratory condition in a human subject by administering a therapeutically effective amount of a first nitrodilator in a first formulation and a second nitrodilator in a second formulation. Nitrodilators cause vasodilation by acting extracellularly to activate the NANOS. Liposome-encapsulated nitrodilators cannot activate the NANOS through extracellular stimulation. However, this formulation of nitrodilators can efficiently load nitrogen oxide moieties into the cells and therefore contribute to the NANOS. In certain embodiments, the nitrodilator is encapsulated in liposomes. In certain embodiments, the first formulation is a sublingual tablet or a trans-lingual spray of nitroglycerin and the second formulation is a liposomal formulation of DNICs, GSNO, or heme-NO. Embodiments include methods of treating a vascular condition or a respiratory condition in a human subject by administering a therapeutically effective amount of a nitrodilator in a first formulation and the same nitrodilator in a second formulation. In certain embodiments, the first formulation is a sublingual tablet or a trans-lingual spray of nitroglycerin and the second formulation is a liposomal formulation of nitroglycerin.

A number of embodiments have been described. Nevertheless, it will be understood that various modifications may be made without departing from the spirit and scope of the description. Accordingly, other embodiments are within the scope of the following claims.

What is claimed is:

1. A method of treating a vascular or a respiratory condition in a subject, the method comprising:
administering to the subject a therapeutically effective amount of a nitrogen oxide compound and a nitrodilator.

2. The method of claim 1, wherein the vascular condition is one or more of a coronary, a pulmonary, or a peripheral arterial disease.

3. The method of claim 1, wherein the nitrogen oxide compound is one or more of a nitro-group containing compound, a nitrate-group containing compound, a nitric oxide (NO) donor, and an endogenous NO producing amino acid.

4. The method of claim 3, wherein the nitro-group containing compound is one or more of a nitrite salt, a D-NG-Nitro arginine methyl ester, and a nitro-group containing low molecular weight molecules, or proteins, or lipids.

5. The method of claim 3, wherein the nitrate-group containing compound is one or more of a nitrate salt or a nitrate-group containing low molecular weight molecules, or proteins, or lipids.

6. The method of claim 3, wherein the NO donor is a NONOate or an hydroxamic acid such as deferoxamine, acetohydroxamic acid, and hydroxyurea.

7. The method of claim 3, wherein the endogenous NO producing amino acid is arginine or citrulline or combinations thereof.

8. The method of claim 1, wherein the therapeutically effective amount of a nitrogen oxide compound ranges from about 1 mg to about 10 g.

9. The method of claim 1, wherein the nitrodilator is nitroglycerin, isosorbide dinitrate, isosorbide mononitrate, nitroprusside, amyl nitrite, a S-nitrosothiol, an heme-NO, or a dinitrosyl iron complex.

10. The method of claim 9, wherein the therapeutically effective amount of nitroglycerin is about 0.1 mg to about 0.6 mg.

11. The method of claim 9, wherein the therapeutically effective amount of isosorbide mononitrate is about 10 mg to about 300 mg.

12. The method of claim 1, wherein the nitrogen oxide compound is a nitrite compound.

13. The method of claim 12, wherein the nitrodilator and the nitrite compound are administered to the subject at a ratio ranging from 1:1 to 1:3333.

14. The method of claim 13, wherein the nitrodilator and the nitrite compound are administered to the subject as a fixed-dose combination.

15. The method of claim 1, wherein the nitrogen oxide compound is a nitrate compound.

16. The method of claim 15, wherein the nitrodilator and the nitrate compound are administered to the subject at a ratio ranging from 1:1 to 1:33333.

17. The method of claim 16, wherein the nitrodilator and the nitrate compound are administered to the subject as a fixed-dose combination.

18. The method of claim 1, further comprising the step of:
administering a therapeutically effective amount of a stimulator of soluble guanylyl cyclase to the subject.

19. The method of claim 18, wherein the stimulator of soluble guanylyl cyclase is riociguat or vericiguat.

20. method of claim 1, further comprising:
administering a therapeutically effective amount of an activator of soluble guanylyl cyclase to the subject.

21. The method of claim 20, wherein the activator of soluble guanylyl cyclase is runcaciguat, cinaciguat, ataciguat, or kynurenine.

22. The method of claim 1, further comprising:
administering a therapeutically effective amount of a cGMP-phosphodiesterase inhibitor to the subject.

23. The method of claim 22, wherein the cGMP-phosphodiesterase inhibitor is sildenafil, vardenafil, or tadalafil.

24. The method of claim 1, wherein the nitrodilator is nitric oxide administered via inhalation.

25. The method of claim 24, wherein the nitric oxide is administered along with a nebulized thiol.

26. The method of claim 25, wherein the nebulized thiol is L-cysteine or glutathione.

27. The method of claim 1, wherein the respiratory condition is apnea, and pulmonary hypertension, or chronic obstructive pulmonary disease.

28. A method of treating a vascular condition in a subject, the method comprising:
administering to the subject a therapeutically effective amount of a first nitrodilator in a first formulation to activate a nitrodilator-activatable intracellular NO store (NANOS) and a second nitrodilator in a second formulation to contribute to the NANOS.

29. The method of claim 28, wherein the first nitrodilator is nitroglycerin and the first formulation is a sublingual tablet or a trans-lingual spray.

30. The method of claim 29, wherein the second nitrodilator is nitroglycerin and the second formulation is a nanoliposome.

31. The method of claim 29, wherein the second nitrodilator is a dinitrosyl iron complex, S-nitrosoglutathione, or heme-NO and the second formulation is a nanoliposome.

32. A method of treating a vascular condition in a subject, the method comprising:
administering to the subject a therapeutically effective amount of a nitrogen oxide compound and a cGMP-phosphodiesterase inhibitor.

33. The method of claim 32, wherein the nitrogen oxide compound is a nitrate-group containing compound or a nitrite compound.

34. The method of claim 32, wherein the cGMP-phosphodiesterase inhibitor is sildenafil, vardenafil, or tadalafil.

35. The method of claim 32, wherein the vascular condition is erectile dysfunction.
